(19) United States
(12) Patent Application Publication
(10) Pub. No.: US 2006/0241418 A1
(43) Pub. Date: Oct. 26, 2006

(54) Electronic endoscope system

(75) Inventor: Kazunori Abe, Saitama (JP)

Correspondence Address: BIRCH STEWART KOLASCH & BIRCH, PO BOX 747, FALLS CHURCH, VA, 22040-0747, US

(73) Assignee: Fujinon Corporation

(21) Appl. No.: 11/392,887

(22) Filed: Mar. 30, 2006

(30) Foreign Application Priority Data
Mar. 31, 2005 (JP) 2005-103231

(51) Int. Cl. A61B 6/00

(52) U.S. Cl. 600/433; 385/117

(57) ABSTRACT

An electronic endoscope system has a personal computer, an adjustment monitor and an adjustment processor unit. The personal computer is for changing parameters related to image quality of an endoscopic image. The adjustment monitor displays an adjustment screen, showing information of the parameters, synthesized on an endoscopic image. The adjustment processor unit is connected to the personal computer and the adjustment monitor. The adjustment processor unit produces adjustment data in a data producing section in accordance with changes of the parameters input from the personal computer and sends the adjustment data from a transmitter to an electronic endoscope. The adjustment processor also receives an imaging signal from the electronic endoscope by a receiver and produces the endoscopic image from the imaging signal in a signal processing section. The adjustment processor then synthesizes the adjustment screen onto the endoscopic image and outputs the synthesized image to the adjustment monitor.

### BACKGROUND OF THE INVENTION

1. Field of the Invention

The present invention relates to an electronic endoscope system constituted of an electronic endoscope, a processor unit and an endoscope monitor.

2. Background Arts

Medical diagnoses using an electronic endoscope are widely performed. The electronic endoscope has a built-in imaging device such as a CCD at a front end of an insertion section, which is inserted into a body cavity. A processor device applies signal processing to the imaging signals obtained with the CCD, and the image inside of the body cavity (endoscopic image) can be observed on a monitor.

When parameters related to image quality of the endoscopic image (parameters related to white balance, color and the like) are adjusted in an electronic endoscope system, an adjustment mode, which is different from a mode for performing endoscopic diagnoses, is executed. In the adjustment mode, information of the parameters is displayed on a monitor, which also displays the endoscopic image, and a cursor on the monitor for changing the parameters is operated with use of a keyboard or a mouse (for example, see Japanese Patent Laid-Open Publication No. 9-113820)

In the electronic endoscope system disclosed in the Japanese Patent Laid-Open Publication No. 9-113820, the adjustment mode is prepared separately from the mode for performing endoscopic diagnoses, and the parameter information is displayed on the monitor, which is for displaying the endoscopic image. Therefore, it has been a problem that the endoscopic diagnosis has to be stopped to adjust the parameters. This problem may be solved by displaying both of the endoscopic image and the parameter information on the monitor at the same time with diving display area for them. However, this configuration leaves another problem like difficulty of observation of the endoscopic image due to restriction of the display area thereof. In addition, the parameters are adjusted by a service person of a maker or the like who is not a medical expert in front of patients. Therefore, this configuration is not preferable in terms of protecting the patients' privacy.

### SUMMARY OF THE INVENTION

An object of the present invention is to provide an electronic endoscope system capable of adjusting parameters related to image quality of an endoscopic image without interfering with an operation of an endoscopic diagnosis.

Another object of the present invention is to provide an electronic endoscope system capable of protecting patients' privacy.

In order to achieve the above and other objects, the electronic endoscope system of the present invention includes an operation input device, an adjustment monitor and an adjustment processor unit. The operation input device is for changing parameters related to image quality of a first endoscopic image. The adjustment processor unit is connected to the operation input device and the adjustment monitor. The adjustment processor unit has a data producing section, a transmitter, a receiver, an endoscopic image producing section and an image processing section. The data producing section produces adjustment data in accordance with changes of the parameters input from the operation input device. The transmitter transmits the adjustment data to the electronic endoscope. The receiver receives an imaging signal from the electronic endoscope. The endoscopic image producing section produces a second endoscopic image from the received imaging signal. The image processing section synthesizes the second endoscopic image and an adjustment screen showing information of the parameters and outputs the synthesized image to the adjustment monitor.

The transmitter transmits the adjustment data by a radio wave, whereas the receiver receives the imaging signal by a radio wave. In this case, the radio wave used for transmitting the adjustment data and the radio wave used for receiving the imaging signal are of different frequency zones that do not interfere with each other. The radio wave used for transmitting the adjustment data is in the frequency zone of 56 kHz, whereas the radio wave used for receiving the imaging signal is in the frequency zone of 1.2 GHz or 2.4 GHz.

In a preferable embodiment of the present invention, the electronic endoscope further includes a signal processing section for applying signal processing to the imaging signal based on the parameters. The signal processing section changes the parameters in accordance with the adjustment data.

It is preferable that the image processing section synthesizes the second endoscopic image and the adjustment screen by overlaying the adjustment screen on the second endoscopic image. It is preferable that the electronic endoscope and the endoscope processor unit communicate the imaging signal each other by a radio wave.

According to the electronic endoscope system of the present invention, the device for adjusting the parameters related to the image quality of the endoscopic image is provided independently from the device used for the endoscopic diagnoses. For this configuration, the endoscopic diagnosis is not interrupted in order to adjust the parameters. In addition, the adjustment of the parameters is performed at a place away from patients so as not to be seen by them. Owing to this, it is possible to protect the patients' privacy.

### BRIEF DESCRIPTION OF THE DRAWINGS

One with ordinary skill in the art would easily understand the above-described objects and advantages of the present invention when the following detailed description is read with reference to the drawings attached hereto.

FIG. 1 is a schematic view illustrating composition of an electronic endoscope system;

FIG. 2 is a block diagram illustrating an electrical structure of an electronic endoscope;

FIG. 3 is a block diagram illustrating an electrical structure of an endoscope processor unit;

FIG. 4 is a block diagram illustrating an electrical structure of an adjustment processor unit; and

FIG. 5 is an explanatory view illustrating a screen displayed on an adjustment monitor.

### PREFERRED EMBODIMENTS OF THE INVENTION

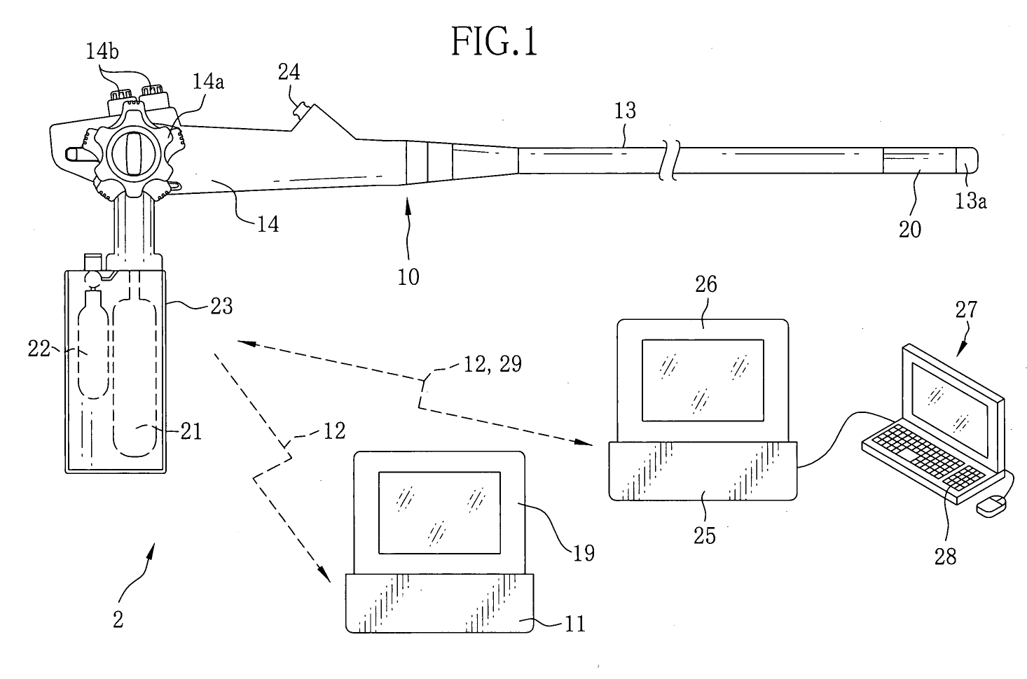

In FIG. 1, an electronic endoscope system 2 is constituted of an electronic endoscope 10, an endoscope processor unit 11, an endoscope monitor 19, an adjustment processor unit 25, an adjustment monitor 26 and a personal computer (hereinafter, PC) 27 as an operation input device. The electronic endoscope system 2 communicates signals between the electronic endoscope 10 and the endoscope processor unit 11 by a radio wave 12 in a first frequency zone (for example, 1.2 GHz) or in a second frequency zone (for example, 2.4 GHz) to which a plurality of channels are allocated. The adjustment processor unit 25, the adjustment monitor 26 and the PC 27 are provided at a place away from patients so as not to be seen from them, such as a part of an endoscopic diagnosis room partitioned from the electronic endoscope 10 and the endoscope processor unit 11, or a different room from the endoscopic diagnosis room.

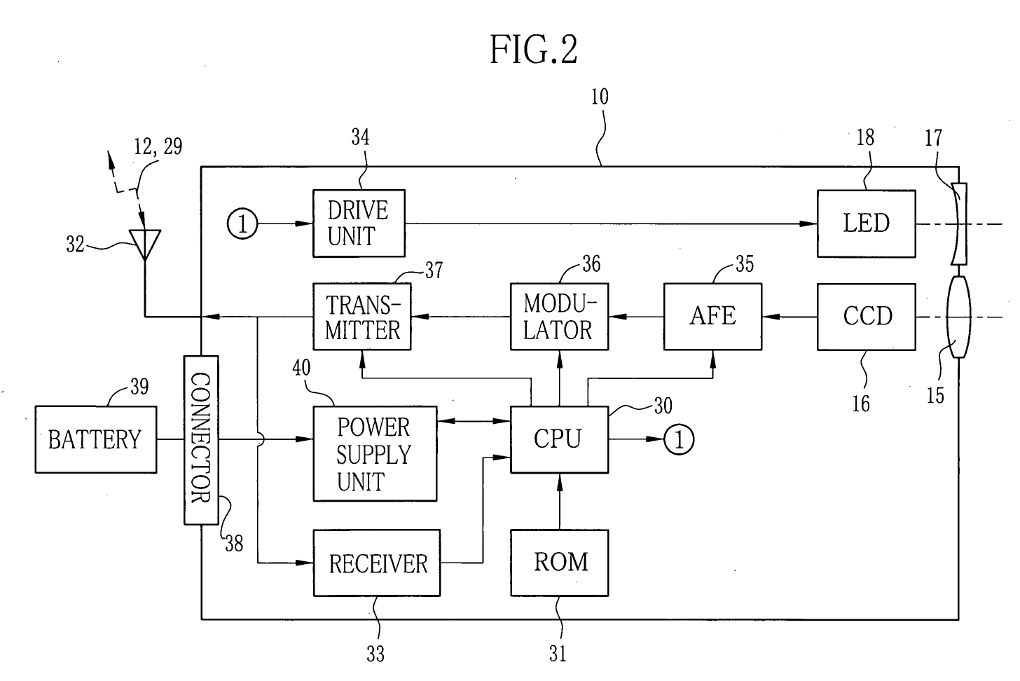

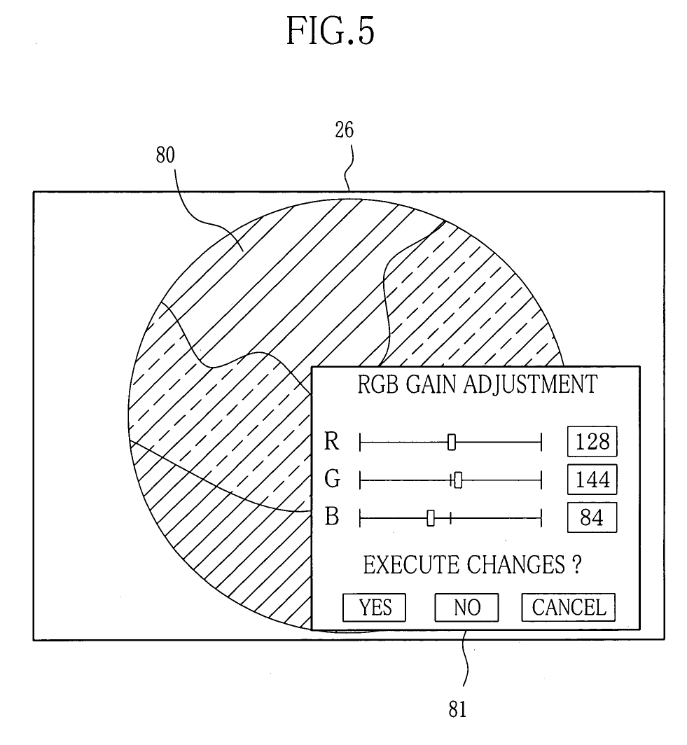

The electronic endoscope 10 is provided with an insertion section 13 inserted into a body cavity and an operating section 14 connected to a base portion of the insertion section 13. An objective lens 15, a CCD 16, an illumination lens 17 and an LED light source (hereinafter, LED) 18 (see FIG. 2) are built in a front end portion 13a provided at a front end of the insertion section 13. The objective lens 15 is for taking image light of a region to be inspected inside the body cavity. The CCD 16 is an imaging element for photographing images of the region to be inspected inside the body cavity. The LED 18 is for illuminating inside the body cavity. The image in the body cavity obtained by the CCD 16 is displayed as an endoscopic image 80 (see FIG. 5) on the endoscope monitor 19 connected to the endoscope processor unit 11.

A curving portion 20 constituted of plural curving pieces jointed together is provided next to the front end portion 13a. A wire extending through inside the insertion section 13 is pushed and pulled by operating an angle knob 14a provided in the operation section 14 to curve and move the curving portion 20 from right to left and up and down so that the front end portion 13a can be directed in any direction inside the body cavity.

A cartridge 23 including a water tank 21 for storing water and an air bottle 22 for storing air is removably attached below the operating section 14. In response to the operation of water/air feeding buttons 14b, the water in the water tank 21 and the air in the air bottle 22 pass through each feed pipe provided in the electronic endoscope 10 to be sprayed out of a cleaning nozzle (not shown) formed in the front end portion 13a toward the objective lens 15. Thereby, foreign matters adhered to a surface of the objective lens 15 is removed and the air is sent inside the body cavity. The cartridge 23 is positioned to be in contact with a wrist of the operator using the electronic endoscope 10 to stabilize the operability of the electronic endoscope 10. Note that the reference numeral 24 represents a forceps opening through which a treatment tool is inserted.

The adjustment processor unit 25 is connected to the adjustment monitor 26 and the PC 27 through cables. The PC 27 has an input section 28 constituted of a mouse and a keyboard. The adjustment processor unit 25 transmits adjustment data in accordance with changes of parameters related to image quality of the endoscopic image 80 (data representing changes of various parameters such as white balance correction, color tone correction, γ-correction and the like) input from the input section 28 as a radio wave 29 to the electronic endoscope 10. In addition, the adjustment processor unit 25 outputs an endoscopic image 80 on which an adjustment screen 81 showing information of the parameters is synthesized (see FIG. 5), to the adjustment monitor 26.

In FIG. 2, a CPU 30 controls the overall operation of the electronic endoscope 10. A ROM 31 storing various programs and data for controlling the operation of the electronic endoscope 10 is connected to the CPU 30. The CPU 30 reads out the necessary program and data from the ROM 31 and controls the operation of the electronic endoscope 10.

The CPU 30 is connected to a receiver 33 that receives the adjustment data as the radio wave 29 from the adjustment processor unit 25 via an antenna 32. The CPU 30 controls the operation of an AFE 35 in accordance with the adjustment data input from the receiver 33.

A drive unit 34 is connected to the LED 18, and turns on/off the LED 18 under the control of the CPU 30. The light from the LED 18 illuminates the region to be inspected inside the body cavity through the illumination lens 17. The LED 18 may be provided in the operating section 14. In this case, the light is guided to the front end portion 13a from the operating section 14 by a light guide.

The image light of the region to be inspected inside the body cavity is focused by the objective lens 15 on an imaging surface of the CCD 16, which outputs an imaging signal corresponding to the image light on each pixel. The AFE 35 applies correlation double sampling, amplification and A/D conversion to the imaging signal from the CCD 16 to convert it into a digital image signal.

A modulator 36 applies, for example, digital orthogonal modulation to the digital image signal output from the AFE 35 to generate an RF signal. A transmitter 37 transmits the RF signal as the radio wave 12 in the first or second frequency zone to the endoscope processor unit 11 and the adjustment processor unit 25 via the antenna 32.

A battery 39 is connected to a connector 38. The battery 39 supplies electric power to each section of the electronic endoscope 10 through a power supply unit 40 controlled by the CPU 30. A battery chamber (not shown) for containing the battery 39 is provided at the rear end of the operating section 14, and the connector 38 is arranged inside the battery chamber.

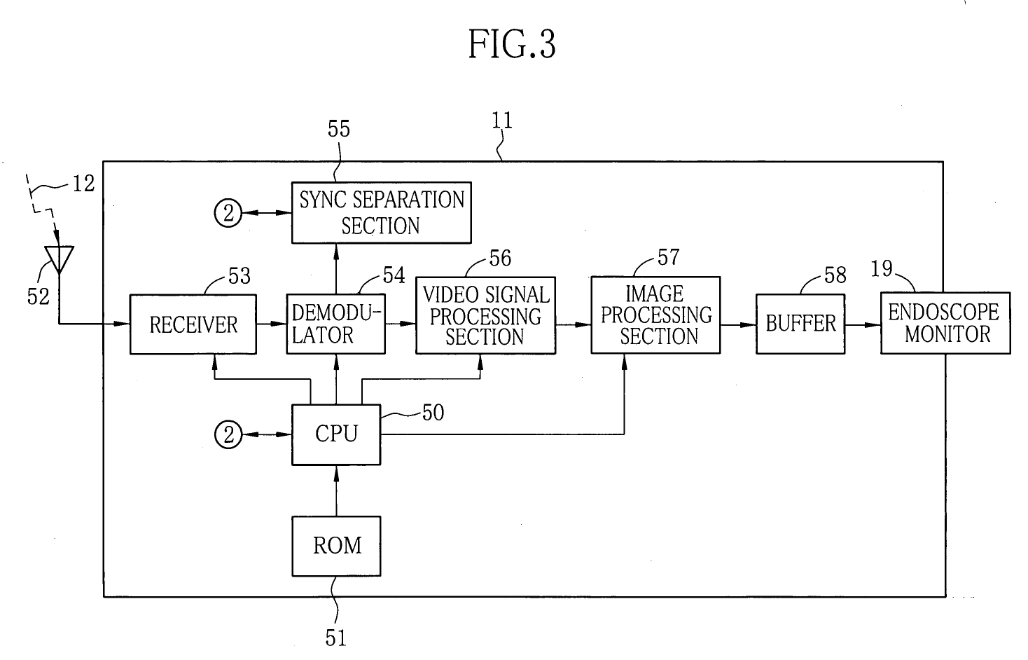

In FIG. 3, a CPU 50 controls the overall operation of the endoscope processor unit 11. A ROM 51 storing various programs and data for controlling the operation of the endoscope processor unit 11 is connected to the CPU 50. The CPU 50 reads out the necessary program and data from the ROM 51 and controls the operation of the endoscope processor unit 11.

An antenna 52 receives the radio wave 12 from the electronic endoscope 10. A receiver 53 amplifies the radio wave 12, that is, the RF signal received by the antenna 52. A demodulator 54 applies, for example, the digital orthogonal detection to the RF signal to demodulate it into the image signal before being modulated in the electronic endoscope 10.

A sync separation section 55 separates a synchronizing signal from the image signal demodulated in the demodulator 54 under the control of the CPU 50 by amplitude separation, and then separates a horizontal synchronizing signal and a vertical synchronizing signal by frequency separation. A video signal processing section 56 produces a digital video signal from the image signal. An image processing section 57 applies various kinds of image processing such as masking and character information addition to the digital video signal produced in the video signal processing section 56. A buffer 58 temporarily stores the video signal to which the various kinds of processing are applied to be displayed as the endoscopic image 80 on the endoscope monitor 19.

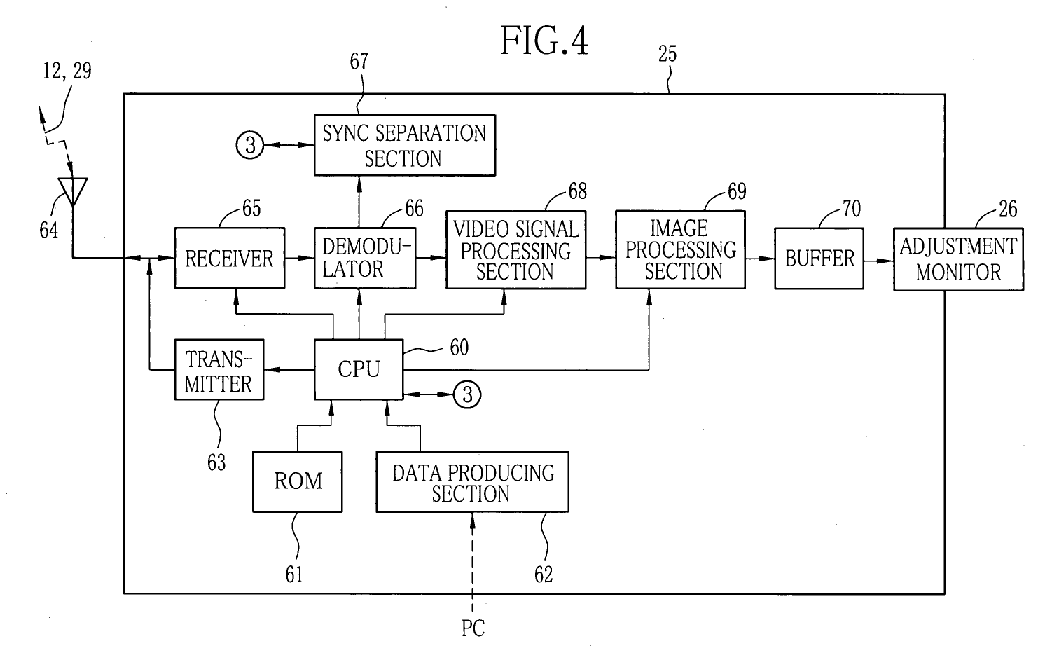

In FIG. 4, a CPU 60 controls the overall operation of the adjustment processor unit 25. A ROM 61 storing various programs and data for controlling the operation of the adjustment processor unit 25 is connected to the CPU 60. The CPU 60 reads out the necessary program and data from the ROM 61 and controls the operation of the adjustment processor unit 25.

The CPU 60 is connected to a data producing section 62. The data producing section 62 produces the adjustment data in accordance with the changes of the parameters input from the input section 28 and sends the adjustment data to the CPU 60. The CPU 60 sends the adjustment data from the data producing section 62 to a transmitter 63.

An antenna 64 receives the radio wave 12 from the electronic endoscope 10. The antenna 64 also sends the adjustment data, which was produced in the data producing section 62, went through the CPU 60 and was modified into the radio wave 29 in the transmitter 63, to the electronic endoscope 10. In order to prevent the radio waves 12 and 29 from interfering with each other, the radio wave 29 is in a different frequency zone from that of the radio wave 12 and it is, for example, of 56 kHz. Note that a receiver 65, a demodulator 66, a sync separation section 67, a video signal processing section 68 and a buffer 70 have the same functions as the receiver 53, the demodulator 54, the sync separation section 55, the video signal processing section 56 and the buffer 58, respectively. Therefore, explanations thereof are omitted.

An image processing section 69 synthesizes the adjustment screen 81 on the endoscopic image 80, that is, the video signal produced in the video signal processing section 68. As shown in FIG. 5, the adjustment screen 81 shows the information of the parameters. For example, gain values of respective RGB colors of the endoscopic image 80 are shown in the forms of both status bars and digits as the parameter information for RGB gain adjustment. In order to perform the RGB gain adjustment, the gain value of each color is changed by moving a slider of the corresponding status bar or by directly inputting numeric value with use of the input section 28. The changes of the parameters are executed by selecting “YES” button. Note that software for adjusting the parameters is installed in the PC 27 therefore the PC 27 displays an adjustment window that shows same information as the adjustment screen 81 on a monitor of the PC 27 in response to the activation of the software.

Next, operation of the above embodiment is explained. When the electronic endoscope system 2 having the above-mentioned structure is used to observe the inside of the body cavity, the insertion section 13 is inserted into the body cavity, and then the image is obtained by the CCD 16 while the LED 18 illuminates the inside of the body cavity to provide the endoscopic image 80 on the endoscope monitor 19.

At this time, the image light of the region to be inspected inside the body cavity is focused by the objective lens 15 on the imaging surface of the CCD 16, and the image signal is output from the CCD 16. The AFE 35 applies the correlation double sampling, amplification and A/D conversion to the image signal output from the CCD 16 to convert it into the digital image signal.

The modulator 36 applies the digital orthogonal modulation to the digital image signal output from the AFE 35 to generate the RF signal. The RF signal is amplified in the transmitter 37 and transmitted as the radio wave 12 from the antenna 32.

In the endoscope processor unit 11, when the radio wave 12 from the antenna 32 of the electronic endoscope 10 is received by the antenna 52, the radio wave 12, that is, the RF signal is amplified in the receiver 53. The demodulator 54 applies the digital orthogonal detection to the amplified RF signal to demodulate it into the image signal before being modulated in the electronic endoscope 10.

The sync separation section 55 applies the synchronizing separation to the image signal demodulated in the demodulator 54 under the control of the CPU 50, and the image signal is output as the digital video signal from the video signal processing section 56. The video signal to which the various kinds of image processing are applied in the image processing section 57 is temporarily stored in the buffer 58 and displayed as the endoscopic image 80 on the endoscope monitor 19.

In the adjustment processor unit 25, when the radio wave 12 from the antenna 32 of the electronic endoscope 10 is received by the antenna 64, the radio wave 12, that is, the RF signal is demodulated by the demodulator 66 into the image signal before being modulated in the electronic endoscope 10. After being applied the synchronizing separation in the sync separation section 67, the image signal is output as the digital video signal from the video signal processing section 68 in the same way as the endoscope processor unit 11.

The video signal output from the video signal processing section 68, that is, the endoscopic image 80 is processed by the image processing section 69 such that the adjustment screen 81 is synthesized thereon and then displayed on the adjustment monitor 26. A user observes the endoscopic image 80 and the adjustment screen 81 displayed on the adjustment monitor 26, and changes the parameters by operating the input section 28 of the PC 27 following the instructions given by the operator of the endoscopic diagnosis.

The adjustment data is produced in the data producing section 62 in accordance with the changes of the parameters input from the input section 28. After going through the CPU 60 and the transmitter 63, the adjustment data is transmitted as the radio wave 29 from the antenna 64 to the electronic endoscope 10.

In the electronic endoscope 10, when the radio wave 29 from the adjustment processor unit 25 is received by the antenna 32, the radio wave 29, that is, the adjustment data is inputted in the CPU 30 via the receiver 33. The AFE 35 is controlled by the CPU 30 to apply various kinds of signal processing to the imaging signal input from the CCD 16 in accordance with the adjustment data. Thereby, the endoscope monitor 19 displays the endoscopic image 80 that reflects the parameter adjustment. Thus, the endoscope monitor 19 only displays the endoscopic image 80 during the parameter adjustment. Therefore, the operation of the endoscopic diagnosis is not interfered.

Since the RF signal and the adjustment data are communicated by the radio waves 12 and 29, respectively, the parameters can be adjusted by remote control. Owing to this, the parameter adjustment can be performed by a service person of a maker who is not a medical expert at a place away from patients so as not to be seen by them, thereby realizing protection of the patients' privacy.

In the above embodiment, the PC 27 is explained as an example of the operation input device. However, it is also possible that the adjustment processor unit 25 is provided with an input device like the input section 28 of the PC 27, and the adjustment processor unit 25 is used as the operation input device. Moreover, in the above embodiment, the adjustment screen 81 is synthesized with the endoscopic image 80 by overlaying the adjustment screen 81 on the endoscopic image 80. However, it is also possible to synthesize these two images such that they are displayed side by side by dividing the display into two. Furthermore, the contents of the parameters and the adjustment screen 81 are merely an example and do not limit the present invention.

In the above embodiment, the electronic endoscope system 2 that communicates signals by the radio waves 12 and 29 is explained as the example. However, the present invention is also applicable to the conventional electronic endoscope systems in which the electronic endoscope and the endoscope processor unit are connected to each other through a signal cable.

In the above embodiment, the electronic endoscope system 2 is explained as it is for medical use, however the present invention is not limited to this. The electronic endoscope system 2 is applicable to other industrial use, such as for photographing images in narrow pipes and the like.

Although the present invention has been described with respect to the preferred embodiments, the present invention is not to be limited to the above embodiments but, on the contrary, various modifications will be possible to those skilled in the art without departing from the scope of claims appended hereto.

What is claimed is:

1. An electronic endoscope system having an electronic endoscope for photographing a region to be inspected of a subject by an imaging device, an endoscope processor unit for producing a first endoscopic image from an imaging signal output from said imaging device, and an endoscope monitor for displaying said first endoscopic image, said electronic endoscope system comprising:
an operation input device for changing parameters related to image quality of said first endoscopic image;
an adjustment monitor; and
an adjustment processor unit that is connected to said operation input device and said adjustment monitor, said adjustment processor unit including
a data producing section for producing adjustment data in accordance with changes of said parameters input from said operation input device,
a transmitter for transmitting said adjustment data to said electronic endoscope,
a receiver for receiving said imaging signal from said electronic endoscope,
an endoscopic image producing section for producing a second endoscopic image from said imaging signal, and
an image processing section for synthesizing said second endoscopic image and an adjustment screen showing information of said parameters and outputting the synthesized image to said adjustment monitor.

2. An electronic endoscope system claimed in claim 1, wherein said transmitter transmits said adjustment data by a radio wave, whereas said receiver receives said imaging signal by a radio wave.

3. An electronic endoscope system claimed in claim 2, wherein said radio wave used for transmitting said adjustment data and said radio wave used for receiving said imaging signal are of different frequency zones that do not interfere with each other.

4. An electronic endoscope system claimed in claim 3, wherein said radio wave used for transmitting said adjustment data is in said frequency zone of 56 kHz, whereas said radio wave used for receiving said imaging signal is in said frequency zone of 1.2 GHz or 2.4 GHz.

5. An electronic endoscope system claimed in claim 1, wherein said electronic endoscope further includes a signal processing section for applying signal processing to said imaging signal based on said parameters, said signal processing section changing said parameters in accordance with said adjustment data.

6. An electronic endoscope system claimed in claim 1, wherein said image processing section synthesizes said second endoscopic image and said adjustment screen by overlaying said adjustment screen on said second endoscopic image.

7. An electronic endoscope system claimed in claim 1, wherein said electronic endoscope and said endoscope processor unit communicate said imaging signal each other by a radio wave.
